(19) United States
(12) Patent Application Publication
(10) Pub. No.: US 2004/0031066 A9
(43) Pub. Date: Feb. 12, 2004

(54) Mouse model for rheumatoid arthritis

(76) Inventors: Denise L. Faustman, Weston, MA (US); Takuma Hayashi, Malden, MA (US)

Correspondence Address: Leon R Yankwich, Yankwich & Associates, 201 Broadway, Cambridge, MA, 02139, US

(21) Appl. No.: 10/181,167

(22) PCT Filed: Jan. 18, 2001
(86) PCT No.: PCT/US01/01483

(51) Int. Cl. A01K 67/027

(52) U.S. Cl. 800/9; 800/18

(57) ABSTRACT

Nonobese Diabetic Mice (NOD mice) that do not develop diabetes may be bred to produce F₁ offspring that develop a condition that closely mimics rheumatoid arthritis (RA) in humans. The RA-like disease in the F₁ mice, designated NOD-RA mice, is similar to human RA in clinical, radiological, histological and serological characteristics. The parents (F₀) and their progeny (F₁) are not diabetic and never develop hyperglycemia, and the parental mice (F₀) do not themselves exhibit any symptoms of the RA-like condition that afflicts some of their progeny. The incidence, penetrance, gender domination, progression, and lifelong exacerbation of symptoms after pregnancy shown in the RA-like condition afflicting NOD-RA mice are all comparable to phenomena observed in the human disease. The NOD-RA mice provide a new spontaneous model of human RA that will be useful for studying rheumatoid arthristis and testing new drugs and reagents for treating or diagnosing the disease.

### FIELD OF THE INVENTION

The present invention pertains to the field of medical research, particularly to the development of mammalian models of human rheumatoid arthritis.

### BACKGROUND OF THE INVENTION

Rheumatoid arthritis (RA) is a common autoimmune disease characterized by joint swelling, deformation and, ultimately, destruction, culminating in severe physical disability. De Graaf et al., in The Epidemiolog of Chronic Rheumatism, Dellgren and Ball, eds. (Blackwell, Oxford, 1963), pp. 446-56; Meenam et al., Arthritis Rheum., 24:544-50 (1981); Gabriel et al., J. Rheumatol, 26:1269-74 (1999); James, Clin Exp. Rheumatol, 17:392-93 (1999). RA is a progressive condition with well-recognized symptoms including symmetrical peripheral joint swelling and synovial inflammation while sparing the axial skeleton; the presence of rheumatoid factor (RF) autoantibodies; increased concentrations of interleukin-6 (IL-6), interleukin-1β (IL-1β), and granulocyte/macrophage colony-stimulating factor (GM-CSF) in serum and synovial fluid; low concentrations of interleukin-ra (IL-ra); and pregnancy-induced disease remission followed by severe postpartum flares, that is, while women with RA commonly undergo remission during pregnancy, the disease returns and may be even more severe and show a new onset or more accelerated course after delivery. See, Turgen, in Immunology and Serology in Laboratory Medicine. 2^{nd} edition, Shanahan ed. (Mosby Year Book, St. Louis, 1996), pp. 387-98; Hirano et al., Eur. J. Immunol, 18:1797-1801 (1988); Wilder et al., Ann. N. Y. Acad. Sci., 876:14-31 (1999); Iijima et al., J. Rheumatol, 26:755-56 (1999); Ostensen, Ann. N.Y Acad. Sci., 876:13143 (1999). In medical research directed to understanding, diagnosing and treating RA, several animal models of the disease have been described, but no spontaneous animal model that closely mimics all the features of the human disease has been discovered (See, for example, Hang et al., 1982. J. Exp. Med., 155:1690-1701; and Kouskoff et al., 1996. Cell, 87:811-822).

Kouskoffet al. report a RA mouse model that exhibits aggressive arthritis, produced by mating a T cell receptor (TCR) transgenic mouse strain with a NOD strain. This RA mouse model is strictly dependent on the presence of the KRN transgene and is characterized by several inherent symptomological features of RA that distinguish it from human RA (hRA), however, including: 100% penetrance, early (i.e., 25-35 days) onset of disease, attack of the distal interphalangeal joints, inflammation of the spine, large excess of myeloid cells over T lymphocytes and plasma cells in the synovial membrane, a total absence of rheumatoid factor (RF) autoantibodies, and a coating of IgG deposits on internal organs. These features result in a more aggressive RA than the RA typically found in humans. Human RA has preferential disease expression in middle-aged females, peripheral disease sparing of the DIP joints, rheumatoid factor autoantibodies, similar peripheral and joint cytokine derangements, etc. The mechanism of development of arhritis-like disease in NOD/TCR mice differs dramatically from that of natural RA expression in humans, limiting the utility of this RA mouse as a model for hRA.

Thus, it would greatly advance discovery research in the field of RA research if a nonhuman mammalian model faithfully exhibiting the same characteristic physical symptoms of hRA could be obtained.

It has now been surprisingly discovered that a particular breed of mouse commonly used in diabetes research, i.e., the nonobese diabetic or “NOD” mouse, can be used to produce offspring that exhibit a physical symptomology closely matching the symptomology of hRA, including: incomplete penetrance with increased incidence in females comparable to that of hRA, disease onset later in life (5-8 months) with exacerbation or early onset due to pregnancy comparable to that of hRA, no inflammation of the spine comparable to that of hRA, and histological and serological profiles comparable to that of hRA.

### SUMMARY OF THE INVENTION

Accordingly, the present invention provides progeny of NOD mice useful as models of rheumatoid arthritis, such as occurs in humans, e.g., that exhibit joint and limb swelling, symmetrical enlargement of peripheral joints with sparing of axial skeleton (e.g., hips, spine); common tendon deformities, such as spontaneous rupture of extensor tendons and Boutonniere deformity; characteristic histological features, such as synovitis, leukocyte invasion, pannus formation, cartilage destruction and bone degeneration; and characteristic serological changes, such as autoantibodies specific for the Fc region of immunoglobulin G (IgG) and increases in proinflamiatory cytokines, especially IL6, in synovial fluid and serum. The occurrence of such symptoms in the mice, furthermore, mimics the human disease in the pattern of progression of the disease and in disease penetration across a population. Moreover, the development of the disease in the mice shows the same disparate penetrance according to gender and the same pregnancy-correlated remission and postpartum exacerbation in females as observed in human RA sufferers.

The present invention also provides a method for preparing mouse models of human rheumatoid arthritis, and methods for using the RA mouse of the present invention, for example, to test potential therapeutics and/or treatment protocols.

### BRIEF DESCRIPTION OF THE DRAWINGS

FIG. 1 illustrates age- and gender-dependent prevalence of RA in F₁ mice from nondiabetic NOD parents.

FIG. 2 (comprised of FIGS. 2a-t) illustrates histological and skeletal abnormalities in NOD-RA mice: the hind feet skeletons of BALB/c (FIG. 2a) and NOD-RA (FIG. 2b) mice were stained with both Alizarin red and Alcian blue; radiological analysis of the hind feet of BALB/c (FIG. 2c) and NOD-RA (FIG. 2d) mice; and histological analysis by staining with hematoxylin and eosin of the ankle (FIGS. 2e, i, o, & q-s), elbow (FIGS. 2f, j, & p), knee (FIGS. 2g & k), and metatarsophalangeal (FIGS. 2h & 1) joints of the hind limbs of BALB/c (FIGS. 2e-h & q), NOD-RA (FIGS. 2i-n & r) and NOD-IDDM (FIGS. 2o, p, s, & t) mice. Magnification of histological micrographs are; X40 (FIGS. 2e, i, & o), X100 (FIGS. 2f-h, j-m, & p-s), and X400 (FIGS. 2n & t).

FIG. 3 (comprised of FIGS. 3a & b) shows serological profiles, segregated by sex (M/F), of BALB/c, NOD, NOD-RA, and NOD-IDDM mice (FIG. 3a); and of RA humans (Patient-RA) and non-RA humans (Healthy), and diabetic humans (Patient-IDDM) (FIG. 3b).

FIG. 4 (comprised of FIGS. 4a-r) illustrates expression of pro-inflammatory cytokine's receptors in the synovial membrane of the ankle joint. Expression of IL6Rα and IL6Rβ in the synovial membrane of the ankle joint of BALB/c (FIGS. 4a-c), NOD-RA (FIGS. 4d-f) and NOD-IDDM mice (FIGS. 4m, n, & q). Ankle joints of 30-week-old female mice were sectioned and stained either with hentatoxylin and eosin (H&E) (FIGS. 4a, d, & m) or with antibodies to IL6Rcα (FIGS. 4b, e, & n), antibodies to IL-6Rα (FIGS. 4c, f, & q) or normal rabbit IgG (FIGS. 4g, j, & p) as a negative control. Expression of IL-IR I and IL-IR II in the synovial membrane of the ankle joint of BALB/c (FIGS. 4h & i), NOD-RA (FIGS. 4k & l) and NOD-IDDM (FIGS. 4o & r). Ankle joints of 30-week-old female mice were sectioned and stained either with antibodies to IL-1R I (FIGS. 4h, k, & o), antibodies to IL-1R II (FIGS. 4i, l, &r).

FIG. 5 (comprised of FIGS. 5a-o) Expression of NF-κBp50, NF-κBp65 and IκBα in the synovial membrane of the ankle joint of BALB/c (FIGS. 5a-e), NOD-RA (FIGS. 5f-j) and NOD-IDDM (FIGS. 5k-o). Ankle joints of 30-week-old female mice were sectioned and stained either with H&E (FIGS. 5a, f, & k) or with antibodies to NF-κBp50 (FIGS. 5b, g, & l), antibodies to NF-κBp65 (FIGS. 5c, h, & m) and antibodies to IκBα (FIGS. 5d, i, & n) or normal rabbit IgG as a negative control (FIGS. 5e, j, & o).

FIG. 6 depicts the frequency of RA in F₁ female mice produced by nondiabetic NOD parents from The Jackson Laboratory versus Taconic. F₁ female progeny are categorized by age (months). Ankle joint thickness was caliper-measured. Individuals with an ankle joint thickness twice that of age-matched control BALB/c mice are indicated by filled symbols.

FIG. 7 (comprised of FIGS. 7a-c) illustrates the pedigrees of mice generated from nondiabetic NOD parental stock (P₁), indicating the incidence of RA expression in progeny of multiple and diverse generations. FIG. 7a records the incidence of RA among the offspring (up to 6 months) of NOD-RA mice bred with nondiabetic NOD mice. FIG. 7b records the incidence of RA among the offspring (up to 6 months) of NOD-RA mice bred with C57BUJ6 mice. FIG. 7c records the incidence of RA among the offspring (up to 6 months) of NOD-RA mice bred with BALB/c mice.

FIG. 8 (comprised of FIGS. 8a-f) illustrates gross morphology of the hind feet of a 21-week-old F₂ female mouse bred from a BALB/c crossed with a C57BLU6 (FIGS. 8a, b, & c), and the hind feet of a 21-week-old female F₂ mouse bred from a NOD-RA crossed with a C57BL/6 (FIGS. 8d, e, & f).

FIG. 9 (comprised of FIGS. 9a-f) provides photographic evidence of RA development and pregnancy effects on RA in NOD-RA mice. Gross morphology of ankle thickness of a 25-week-old BALB/c female (FIG. 9a); a 25-week-old NOD-IDDM female (FIG. 9b); a 25-week-old NOD-RA female before pregnancy (FIG. 9c), and 8 weeks after delivery and at an age of 33 weeks (FIG. 9d). Ankle thickness is shown before (FIG. 9e), and after (FIG. 9f) pregnancy of F₂ progeny of NOD-RA bred with C57BL/6 (NOD/C57BL-RA), Arrows indicate maximal swelling and joint abnormalities.

FIG. 10 (comprised of FIGS. 10a-b) provides graphic data tracking RA development across pregnancy in F₁ NOD-RA females and BALB/c females, compared to virgin F₁ NOD-RA females (FIG. 10a). Also illustrated are the data for RA development in across pregnancy in F₁ NOD/C57BL-RA females compared to virgin F₁ NOD/C57BL-RA females (FIG. 10b). Data are means (±s.e.) from five animals in each group.

### DETAILED DESCRIPTION OF THE INVENTION

The present invention relates to a method for preparing mice that exhibit the physical characteristics (e.g., one or more symptoms) of human patients suffering from rheumatoid arthritis (RA) and thus provides a murine model of human rheumatoid arthritis. The mice prepared in accordance with the teachings herein are useful in studying the manifestation, penetration and progression of rheumatoid arthritis as occurs in humans, and the mice are also useful for testing new methods and products for the diagnosis and/or treatment of rheumatoid arthritis. The mouse model will be particularly useful for screening new drug candidates for effectiveness against RA.

As used herein, “symptoms” or “characteristics” of rheumatoid arthritis (RA) or RA-like disease refers to any one or more pathological manifestations of rheumatoid arthritis known in the art. Such manifestations may be overt physical characteristics (e.g., joint or limb swelling, or other physical deformities detectable upon physical examination of an individual), or clinical symptoms (e.g., radiological, histological, and serological profiles indicative of RA).

The invention is based on the discovery that progeny of “nondiabetic nonobese diabetic mice” (also known in the art and referred to herein as “nondiabetic NOD mice”, “survivor NOD mice”, or “diabetes-free NOD mice”), develop the classical symptomology of rheumatoid arthritis by 20-28 weeks (or by 12-16 weeks after pregnancy). Moreover, the manifestation of the disease in the progeny was confirmed by clinical (e.g., radiological, histological, and serological) data that closely mimicked similar measurements in human RA patients.

As used herein and consistent with the plain meaning of the term as used in the art, “progeny” refers to any offspring or descendents of an individual. Progeny may refer to one or more individuals, and may span several filial, or “F”, generations (e.g., F₁, F₂, F₃, etc.) generated, either directly or by descent, from parental, or P₁, stock.

As used herein, “mating” or “breeding” refers broadly to the propagation of individuals under controlled conditions to produce progeny. Although a preferred method for breeding, physical mating (i.e., copulation) of a sexually mature male and a sexually mature female, is not an essential feature of the present invention. Any form, method, or mode of reproduction of an individual (e.g., natural or artificial, sexual or asexual) is within the scope of the present invention. Methods such as artificial insemination and embryo implantation, for example, are known to practitioners in the art to provide greater control over zygote selection, to ensure successful fertilization, and to avoid possible injury to the parental stock. In addition, methods of asexual reproduction and/or animal cloning are known in the art and available technologies useful for breeding to produce the RA mouse model of the present invention.

Nonobese diabetic mice (NOD mice) are well known animal subjects for the study of diabetes and other autoimmune diseases that affect humans. The mice are available commercially from several sources (e.g., The Jackson Laboratory, Bar laarbo, Maine). NOD mice were first identified as developing spontaneous autoimmune diabetes in 1980 on the basis of cataract development that is a secondary characteristic of diabetes in humans. Makino et al., Jikken Dobutsu Exp. Animals, 29:1-13 (1980). Ninety percent (90%/) of female NOD mice will develop autoimmune diabetes by 20 to 30 weeks of age; and about 20% to 40% of male NOD mice develop autoimmune diabetes. The diabetes that develops in NOD mice is lethal, and without insulin treatment NOD mice that become diabetic die within about 48 hours, with blood glucose concentrations of approximately 600 mg/deciliter. With insulin treatment, the diabetic mice can survive for several weeks, but the mortality rate in spite of insulin treatment is still approximately 50% at two weeks and uniformly lethal at four weeks.

NOD breeding colonies are commonly maintained by paired matings initiated at 4 to 6 weeks of age and continued until the animals are 20 to 22 weeks of age. This corresponds to the period of maximum breeding potential prior to the usual onset of lethal diabetes. Surviving animals usually are killed after breeding. Pozzilli et al., Immunol Today, 14:193-96 (1993).

A mouse of the present invention that exhibits one or more symptoms of rheumatoid arthritis requires breeding a nondiabetic NOD mouse to produce an arthritic progeny (i.e., a NOD-RA mouse). No incidence of RA symptoms developing in nondiabetic NOD parents has been observed.

When selecting parental stock (i.e., the parental generation, or P₁) for the method of the invention, at least one individual must be nondiabetic NOD, that is, a NOD mouse that, although bred for the development of diabetes, fails to develop autoimmune diabetes. Preferably the nondiabetic NOD individual is female, more preferably both parents will be nondiabetic. Nondiabetic NOD parental stock should be diabetes-free, or euglycemic. Onset of diabetes in mice is signaled by marked hyperglycemia, i.e., rising and remaining above 250 mg/deciliter. Since the onset of diabetes is quickly fatal in NOD mice, the nondiabetic NOD animals useful for the purposes of the present invention are aptly referred to as survivors. Although not essential, in order to be sure that NOD mice selected for the purposes of the present invention are nondiabetic mice that will not develop diabetes, it is preferable that the parental mice selected be at least 12 weeks of age. Preferably the parental mice selected will be 20 weeks old or older, more preferably 22 weeks old or older, most preferably at least 24 weeks old. It is also preferred but not critical that the male and female parental stock be age-matched.

Nondiabetic NOD P₁ mice and their progeny are truly nondiabetic; that is, they are not slow-onset diabetic mice that are destined to develop the disease later but fail to exhibit onset by the typical onset age of 20-30 weeks. None of the diabetes-free P₁ mice or their progeny studied in the examples that follow died or developed detectable glucosuria during an additional ten months of observation. Furthermore, histological examination of their pancreases revealed no occult disease, i.e., no lymphocytic infiltration. Thus, the RA mouse of the invention (also referred to as NOD-RA), as well as its survivor NOD parental stock are not diabetic mice useful for the study of autoinunune diabetes (i.e., the parental nondiabetic NOD mouse and the progeny NOD-RA mouse of the present invention are diabetes-free), and the present invention advantageously provides an alternative to destruction of nondiabetic survivors.

Although maintaining NOD stock past the normal age of diabetes onset is a preferred way of isolating survivor (nondiabetic) NOD mice, it is not necessary to wait 20-30 weeks before practicing the present invention. Since the nondiabetic NOD mice are, as mentioned above, born non-diabetic and will remain free of diabetes, breeding NOD mice without pre-determining whether or not they are nondiabetic may be done, with the development of NOD-RA offspring confirming after the fact that at least one parental mouse was nondiabetic.

Although not critical, prediabetic screening of the mice is helpful in identifying nondiabetic NOD mice useful as parental stock in the practice of the present invention. Screening for glucose levels exceeding 250 mg/dl has been mentioned above, and additional pre-onset diabetic indicators include the appearance of autoantibodies against islets or insulin, the appearance of rheumatoid factor (RF), and increasing IL-6 levels. Although it is not usually practical to assay in mice, lymphocytic infiltration of the pancreas is also an indicator of diabetes onset, and therefore indications that the pancreas of a NOD mouse remains free of infiltrating lymphocytes also identifies a suitable P₁ mouse for the purposes of the invention.

The P₁ matings of nondiabetic NOD pairs consistently produces F₁ mice that show high penetrance of RA symptomology by 20-28 weeks of age. Appearance of characteristic RA symptoms occurs much sooner in F₁ females that become pregnant (e.g., 10-16 weeks after pregnancy).

The F₁ mice exhibit visible bilateral swelling of the ankles and the metatarsophlangeal and proximal interphalangeal joints of both hind feet. No swelling is typically observed at the distal interphalangeal joints. This pattern of joint swelling mimics the pattern characteristic of human RA.

The NOD-RA mice not only model the symptoms of human RA in overt physical characteristics, but also exhibit RA abnormalities upon clinical (e.g., radiological, histological, and serological) examination that closely resemble the symptoms observed in human RA patients. In addition, the penetrance of the RA-like disease in NOD mice is incomplete, as it is in humans, with identical twins of RA sufferers having less than 25% incidence lifelong of also developing RA. Also, there is a marked predominance (85%) of females in a population of F₁ mice that develop the RA disease, which is comparable to the predominance (75% females) found in human RA patients.

Not all F₁ mice will be NOD-RA mice (see Examples, infra.), and NOD-RA mice will usually begin to exhibit RA symptoms such as ankle swelling by 12 weeks of age, with peak onset at about 22-28 weeks of age.

The NOD-RA mice according to the invention may be used in any application where a mammalian model of human RA would be advantageous. The NOD-RA mice prepared according to the invention will be especially useful in testing RA diagnostic reagents, such as antibodies, dyes or magnetic resonance imaging agents, or in screening potential therapeutic agents. Many applications for such NOD-RA mice will immediately suggest themselves to persons skilled in this field.

A particularly useful application for the mouse RA model of the invention will be in drug screening. A method of screening utilizing NOD-RA mice according to the invention will comprise the step of administering to a NODRA mouse showing symptoms of RA a candidate drug for the treatment of RA and then analyzing the mouse for improvement in any of the symptoms of the disease, e.g., lowering of RA or IL-6 levels, remission or decrease in, e.g., joint swelling, abnormal joint flexion or hyperextension, etc. Any favorable change in RA symptomology in the NOD-RA mouse receiving the candidate drug will indicate that the candidate drug is useful for treating RA.

Another particularly useful application of the mouse RA model of the invention will be in screening for preventive medicines. Since the RA-like disease in NOD-RA mice has the same penetrance and gender and age correlation as in human RA patients, a good method for testing candidate RA preventive medicines will comprise administering a candidate medicine to the progeny of a nondiabetic NOD mouse, then observing whether the expected penetrance of the disease in the progeny is altered in response to receiving the candidate medicine. If the incidence of appearance of RA in the progeny is lower than expected, if the severity of the disease is reduced, or if the onset of RA is delayed, then this indicates that the medicine has therapeutic properties.

The NOD-RA mouse according to the invention is further described in the examples that follow. These examples are illustrative of the methods and the mouse of the invention, and are not intended to limit the concept of the invention in any way.

### Example 1

### Generation and Physical Determination of NOD-RA Maice

Ten surviving diabetes-free (euglycemic) NOD females at 24 weeks of age were mated with six age-matched diabetes-free NOD males. 175 F₁ offspring were categorized by monitoring for ankle swelling. Twenty-eight of 87 female (32%) F₁ offspring and six of 88 male (7%) F₁ offspring of all ages exhibited ankle swelling. F₁ progeny exhibiting visible joint swelling were designated NOD-RA mice. Comparative colonies of BALB/c mice, parental P₁ NOD mice, and a cohort of ten new NOD females obtained from Jackson Laboratories were maintained as controls. None of the control groups exhibited particular swelling and appeared to have normal morphology of the peripheral limbs under similar aging and housing conditions.

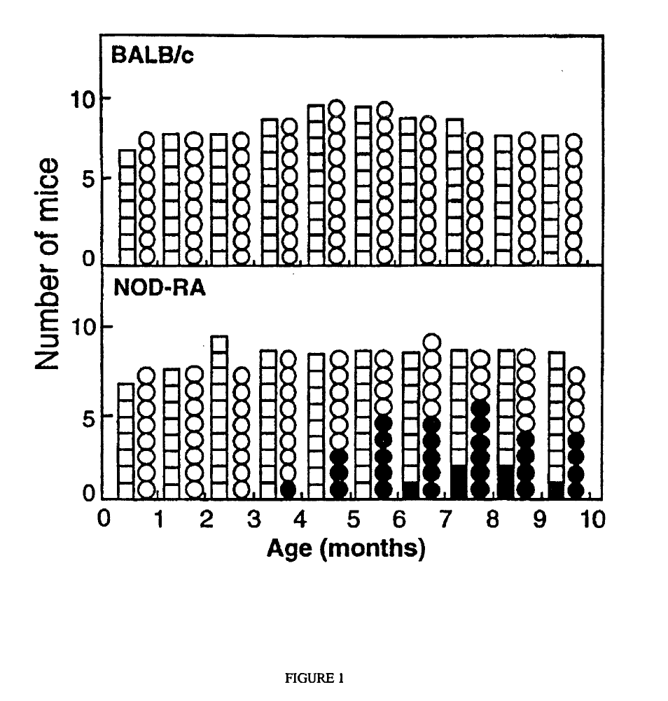

FIG. 1 depicts statistical analyses of prevalence of RA in F₁ progeny of nondiabetic NOD parents, by compiling data on all F₁ progeny that were not sacrificed for histological analysis before 10 months of age. All mice were categorized by age and had ankle thickness measured using calipers. Those mice with an ankle thickness at least twice that of age-matched BALB/c controls are indicated by filled symbols (574, 570). Females are indicated with circles (603, 574 ) and males are indicated with squares (570, 574). The onset of presumed RA was first apparent at 3 months, with a peak onset at 7 months among NOD-RA females. The incomplete penetrance of the murine RA-like condition resembles that of human RA; for genetically identical human twins in which RA affects one twin, the other twin has a <25% lifelong chance of developing this condition. Furthermore, the predominantly female expression of the mouse RA-like disease (82%) is similar to that observed for human RA (83% of RA patients are female).

### Example 2

### Radiological and Histological Analyses of NOD-RA Mice

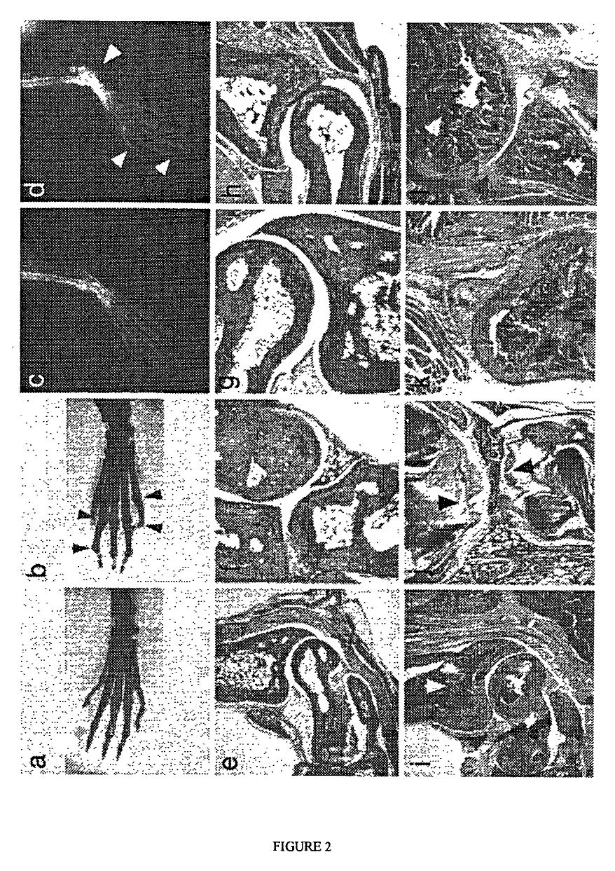

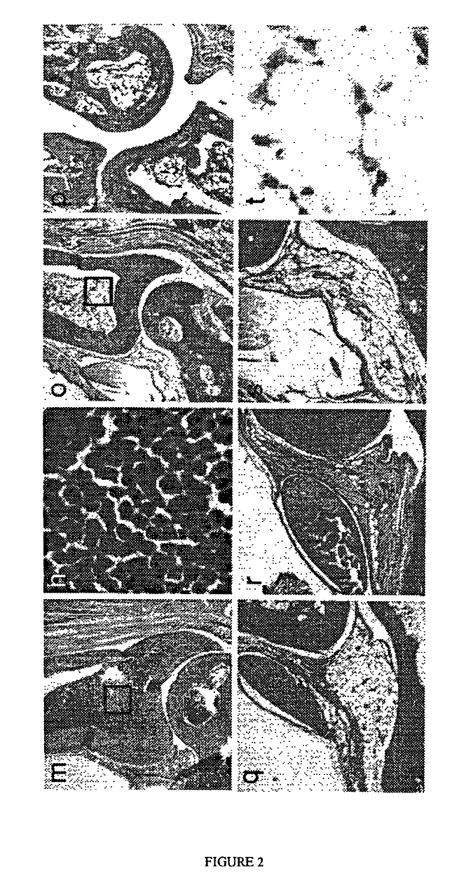

Skeletal examinations were made of NOD-RA mice and BALB/c controls at 7 months of age or older to characterize joint disease occurring in the former (see FIG. 2). Hind feet skeletons of BALB/c (FIG. 2a) and NOD-RA (FIG. 2b) mice were stained with Alizarin red and Alcian blue. Comparison of the NOD-RA and BALB/c skeletal specimens shows enlargement of the peripheral synovial joints and severe joint destruction in the NOD-RA animals. Arrowheads (FIG. 2b) indicate tendon rupture secondary to joint subluxation. Radiological analysis of the hind feet of BALB/c (FIG. 2c) and NOD-RA (FIG. 2d) mice shows obliteration of the ankle joint space, central osseous erosion of the ankle, and fusiform swelling of soft tissue extending from the ankle down to the digits of the NOD-RA animal. Arrowheads indicate the locations of the radiographic deformities. Radiological and skeletal findings demonstrated that, in contrast to the peripheral joints, the sacroiliac and vertebral joints in NOD-RA mice were not affected.

Histological micrographs of ankle, elbow, knee and metatarsophalangeal joints were also prepared by fixing in 4% paraformaldehyde, decalcified for 8 h in 6% nitric acid, and incubated for 8 h with 4% paraformaldehyde, and embedded in paraffin. Sections (5 μm) were prepared and stained with hematoxylin and (FIGS. 2e-l). For skeletal preparations, double staining for cartilage and bone was performed with Alizarin red and Alcian blue (respectively).

FIGS. 2e-h, and q were prepared from a BALB/c mouse, FIGS. 2i-n, and r were prepared from a NOD-RA mouse, and FIGS. 2o, p, s and t were prepared from a NOD-IDDM mouse. Ankle preparations are shown in FIGS. 2e, i, o, & q-s; elbow preparations in FIGS. 2f, j, & p; knee preparations in FIGS. 2g and k; and metatarsophalangeal joint preparations in FIGS. 2h and l. The joint histology of the NOD-RA animals is characterized by pannus proliferation and invasion of the articular space (green arrowhead in FIGS. 2i and l), degradation of articular cartilage (dark arrowheads in FIG. 2j), osseous erosion, and infiltration of inflammatory cells at the ends of the bones (yellow arrowheads in FIGS. 2i and l). A multinucleated giant cell associated with bone and cartilage detritus is also apparent (orange arrowhead in FIG. 2k).

From radiological and histological evidence, it was seen that the disease was more prominent in the hind limbs than in the fore limbs and exhibited a distal-to-proximal gradient of severity, with general sparing of the distal interphalangeal joints of the limbs. The disease was bilaterally symmetrical in each mouse that developed the RA-like condition. In addition, examination of the spine and hips showed that the disease appeared to spare the axial skeleton, another phenomenon paralleling human RA. Knee joints exhibited a hypertrophic, edematous synovial membrane extending over the femoral condyles and into the intercondylar notches. As in the hands and feet of humans with advanced rheumatoid arthritis, spontaneous rupture of the extensor tendons of the fore and hind feet of the NOD-RA mice was common. Joint malalignment and subluxation were also apparent in skeletal preparations of the NOD-RA mice.

Boutonniere deformity, a common type of tendon rupture and joint deformity in humans with RA, is characterized by hyperextension at the metacarpophalangeal joint and flexion at the proximal interphalangeal joint. NOD-RA mice exhibited virtually identical tendon deformities in their feet, with rupture of extensor tendons with hyperextension at the metatarsophalangeal joint and flexion of the proximal interphalangeal joint.

In human RA, pannus formation in the early stages is confined to the margins of the joints but extends toward the joint capsule as the disease progresses, crosses the synovial and destroys the underlying synovial cartilage. A similar progression is evident in the elbow points of NOD-RA animals (see FIG. 2j). Marrow inflammation and replacement of subchondral bone were evident in the long bones of the ankle (e.g., FIG. 2i), in the knee joint (e.g., FIG. 2k), and in the metatarsophalangeal joint (e.g., FIG. 21) of the hind limbs of NOD-RA mice.

Examination of the pancreas of NOD-RA mice showed normal, well-granulated islets, with no evidence of lymphocyte invasion or surrounding lymphocytes, confirming that the NOD-RA mice were diabetes-free.

### Example 3

### Serological Profiles of NOD-RA Mice

In human rheumatoid arthritis, onset of the disease is also characterized by serological phenomena. See, e.g., Jackson, in Clinical Laboratory Medicine, Tilton, ed. (Mosby Year Book, St Louis, 1992), pp. 485-504. For example, about 70%-90% of RA patients produce “rheumatoid factor” (RF), which comprises autoantibodies specific for the Fc region of immunoglobulin G. In addition, markedly increased concentrations of the proinflammatory cytokine IL-6 are observed in the synovial fluid and serum of RA patients. Diverse patterns of autoantibody production have also been observed in humans with various autoimmune diseases including RA, such as antibodies to double stranded DNA (anti-dsDNA), although anti-dsDNA antibodies are usually a more sensitive marker in humans for lupus erythematosus than for RA.

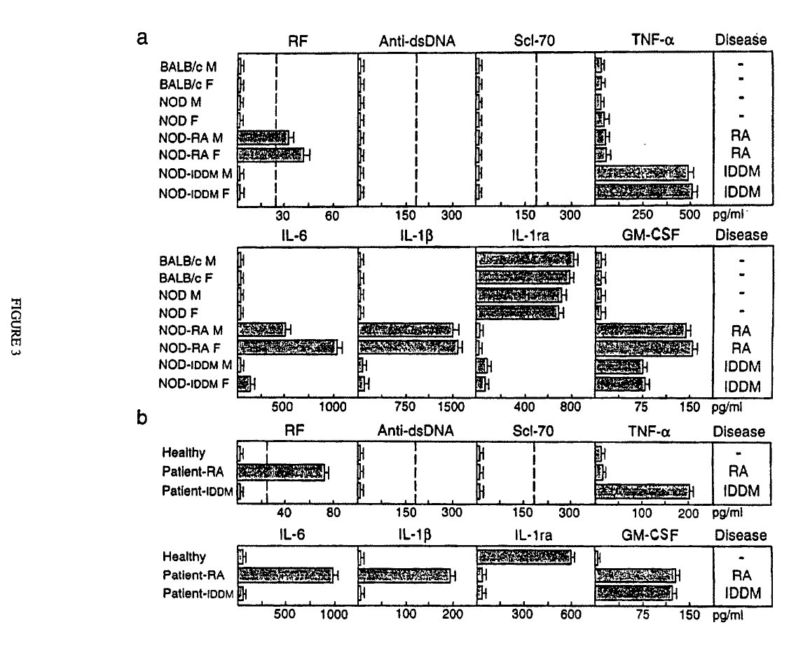

To compare the serological profile of RA patients and NOD-RA mice, sera were assayed in male and female BALB/c mice (controls), NOD mice (controls), NOD-RA mice, NOD mice with insulin-dependent diabetes mellitus (NOD-IDDM), as well as healthy human donors, diabetic human donors, and RA human donors for RF, anti-dsDNA, Scl-70, TNFα, IL-6, IL-1β, 1Lira, and GM-CSF(see FIG. 3).

Human autoimmune serum was obtained from outpatients at the Massachusetts General Hospital (Boston, Mass.). Various commercially available immunoassays were used to quantify human dsDNA antibodies (e.g., Sigma, MO; Product no. EIA503B, standardized against WHO serum, WO/80), human anti-Scl-70 antibodies (e.g., Sigma, MO: product no. EIk549B), and human antibodies to rheumatoid factor IgM (e.g., Sigma, MO: product no. EIA507B). Human GM-CSF, IL-1β, IL-1ra, ILK and TNF-α were also quantified using commercially available ELISA kits (e.g., R&D Systems, MN; product nos. DGM00, NFL80, DRA00, and NF60, and BD PharMingen, Calif.; product no. 2637KI).

To analyze mouse samples, commercially available human immunoassays (e.g., those from Sigma, Mo.) were modified to quantify murine autoantibodies to dsDNA, Scl-70 and RF. For murine anti-dsDNA and murine anti-Scl-70, antibodies to murine IgG labeled with a horseradish peroxidase (HRP) conjugate (Santa Cruz, Calif.; product no. sc-2005) was used as the developing reagent. For the murine RF assay, mouse IgG (Santa Cruz, Calif.; product no. sc-2025) was immobilized onto multi-well plates. Dilute test samples were added to the coated wells and incubated for at least 4 h at room temperature. After washing to remove unbound material, HRP conjugate anti-mouse IgM (Santa Cruz, Calif.; product no. sc-2064) was added to the wells. The wells were washed and incubated with substrate (TMB) and quantified (absorbency at 450 nm). Murine GM-CSF, IL-1β, IL-1ra, IL-6, and TNF-α was quantified using ELISA kits specific for murine cytokines (R&D Systems, MN; product nos. MGM00, MKB00, DRA00, and M6000, and BD PharMingen, Calif.; Product number 2673KI. Note that immunoassay kit DRA00 is useful for both human and murine IL-1ra assays).

Results are presented in FIG. 3. The data presented are means (±s.e.) of values from five subjects for each group. Abnormally high concentrations for RF (>25 IU/ml) and anti-dsDNA (>180 IU/ml) are indicated on the graphs by dotted lines.

RA is characterized by changes in serological parameters; about 70-90% of humans with RA produce RF as a diagnostic maker fbr autoimmunity, which comprises autoantibodies specific for the Fe region of immunoglobulin G. Of the mice examined, only NOD-RA animals (both male and female) produced RF at levels approaching that apparent in human RA patients.

Antibodies to double-stranded DNA (anti-dsDNA) and antibodies to Scl-70 antigen were not detected in the serum of any of the subjects examined. Such antibodies are not a sensitive marker for diagnosis of RA in humans, being typically more specific for systemic lupus erythematosus and other autoimmune disease.

Dysregulation of pro-inflammatory cytokines frequently tracks with clinically distinct autoimmune disease and could be causal to both murine and human disease. For example, increased basal levels of pro-inflammatory cytokine TNF-α are frequently detected in the serum of diabetic NOD mice and autoimmune diabetic humans. The basal expression levels of TNF-α were increased 11-fold exclusively in NOD-IDDM mice; TNF-α levels were normal in NOD-RA, NOD mice without any autoimmune disease expression and BALB/c mice. Similarly, basal TNFA levels were exclusively elevated in diabetic humans; TNF-α levels were normal in screened RA patients

The pro-inflammatory cytokine IL-6 was elevated in the synovial fluid and serum of humans with RA. Among the mouse groups, only NOD-RA mice exhibited high serum concentrations of IL-6 comparable to RA humans; NOD-IDDM, NOD and BALB/c mice had normal IL-6 levels.

IL-1β is an essential inducer of IL-6 expression; high IL-1β serum levels are typical of random humans with autoimmune arthritis but not healthy controls nor diabetic patients. This remarkable trend of reciprocal cytokine expression paralleling human target organ selection was again mirrored in the tightly controlled and known genotypically identical NOD-RA and NOD-IDDM mice. IL-1β expression levels were markedly increased 100-fold in NOD-RA compared with BALB/c mice; IL-1β expression levels were up-regulated only 5-fold in NOD-IDDM compared with BALB/c.

Clinical studies show the beneficial effect of IL-1 receptor antagonist (IL-1ra), a competitive inhibitor of IL-1β, as a biological agent on the rate of joint erosion; autoimmune humans with RA more so than IDDM have suppressions of serum IL-1ra. IL-1ra^{-/-} mice on select genetic background have high IL-1β and IL-6 levels and demonstrate autoimmune inflammation with a RA-like disease. Examination of these phenotypically distinct but genotypically identical autoimmune mice revealed dramatic down-regulation of IL-1ra levels in NOD-RA mice; NOD-IDDM mice to a lesser degree also had suppressed IL-1ra levels.

IL-1 is known to induce various inflammatory cytokines, such as GMCSF. The expression of GM-CSF was elevated in both NOD4,DDM and NOD-RA mice, thus GM-CSF is unlikely a central cytokine determining target specificity.

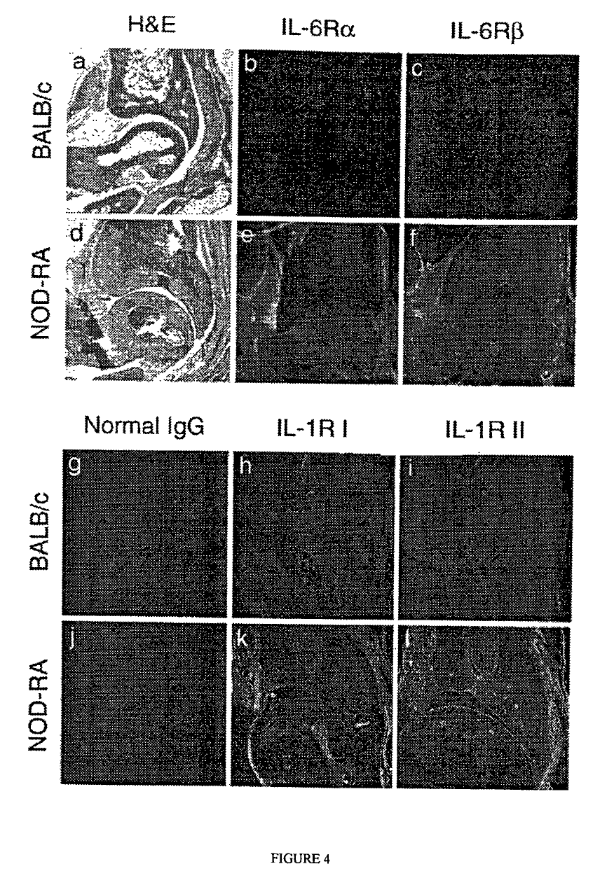

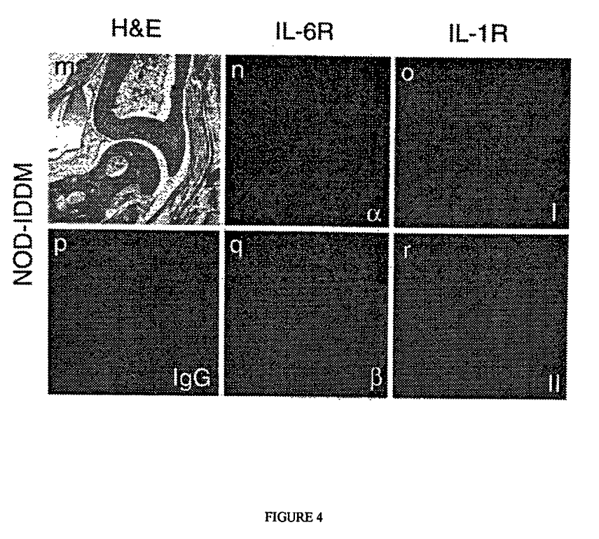

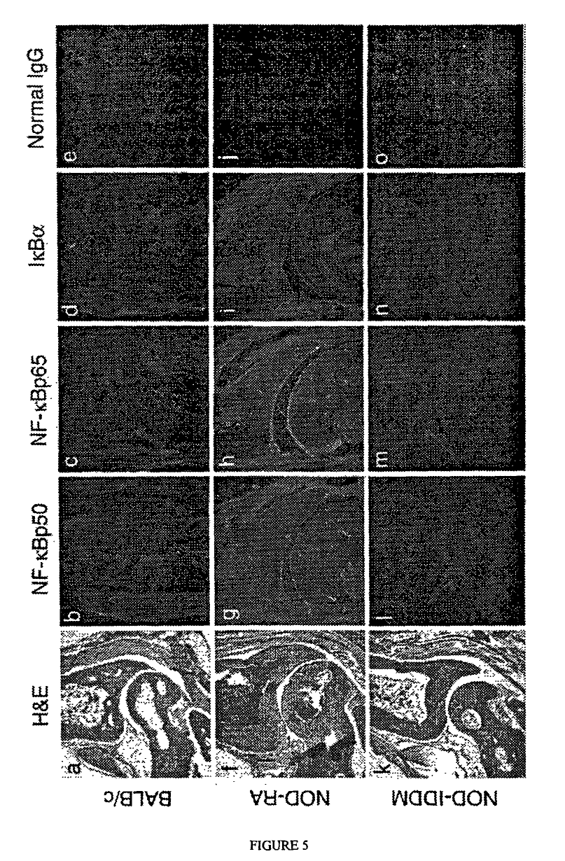

In addition, Immunohistochemical analysis of ankle joint sections from BALB/c (FIGS. 4a-c), NOD-RA (FIGS. 4d-f) and NOD4DDM (FIGS. 5m, n, and q) mice with antibodies to the IL-6 receptors (IL-6Rcα) and β (IL-6Rβ) chains, show marked expression of both IL-6R subunits in only NOD-RA mice (FIGS. 4a-f). The abnormal synovial and subchondrial bone of NOD-RA mice was highly immunofluorescent (FIGS. 4e-f), whereas only a few cells expressed these subunits in the normal synovial membrane of BALB/c mice (FIGS. 4b-c) and NOD-IDDM (FIGS. 4n and q).

Immunohistochemical analysis was also performed on joint sections from BALB/c and NOD-RA mice with antibodies to the IL-1 receptor type I (IL-1RI) and IL-1 receptor type II (FIGS. 5h, i, k, l, o, and r). Marked expression of both IL-1R subunits was apparent in association with the abnormal synovial and sub-chondrial bone inflammation membrane of only NOD-RA mice (FIGS. 5k-i), whereas only a few cells expressed these subunits in the normal synovial membrane of BALB/c mice (FIGS. 5h-i) or NOD-IDDM mice (FIGS. 5o and r).

The transcription factor NF-κB activation in the arthritic joint has been associated with RA progression; it is strongly activated by an IL-1 mediated signal. Therefore, NF-κB subunits, p50, p65 and IκBα are markedly expressed in the abnormal synovial and sub-chondrial bone inflammation membrane of only NOD-RA mice (FIGS. 5f-j), non expression is detected in BALB/c mice (FIGS. 5a-e) or NOD-IDDM mice (FIGS. 5k-o).

### Example 4

### Genetic Basis for RA occurrence in NOD-RA Mice

Because diverse bacterial and viral infections are known to eliminate autoimmune diabetes, and to test the possibility that the generation of RA mice of the present invention is an anomaly attributable to some infective agent and/or pathogen, freshly ordered (The Jackson Laboratories) and similarly housed NOD mice were maintained under conditions identical to those used in Example 1.

The cumulative incidence of diabetes by 30 weeks in these females was consistently around 71% (24 of 34 NOD females). The number of male NOD mice affected by 30 weeks of age was about 28% (9 of the 32 NOD male). These frequencies are consistent with previously reported results. None of these animals developed RA-like disease. It is, therefore, unlikely that RA-like disease expression results from an elusive infective agent and/or pathogen, but is rather genetically determined.

### Example 5

### Specificity of NOD Parental Stock In the Generation of NOD-RA Mice

To demonstrate that the generation of RA mice of the present invention is reproducible and not an anomaly attributable to, for example, the breeding of a particular strain of nondiabetic NOD mouse, experimental protocols described in Example 1 were repeated using newly acquired NOD mouse cohorts from The Jackson Laboratory (Bar Harbor, Me.) and from Taconic (German town, N.Y.) and using newly acquired BALB/c mouse cohorts (The Jackson Laboratory, Bar Harbor, Me.).

Commercially obtained NOD mice were reared for 20-22 weeks, thus selecting for nondiabetic NOD mice as parental stock (P₁). Nondiabetic NOD mice, as well as BALB/c mice (as a control group), were inbred to produce F₁ progeny.

F₁ progeny were grouped into age-matched cohorts. Ankle joint thickness was measured to indicate the occurrence of RA in F₁ mice. Individuals with an ankle joint thickness twice that of age-matched control BALB/c mice were identified as RA mice.

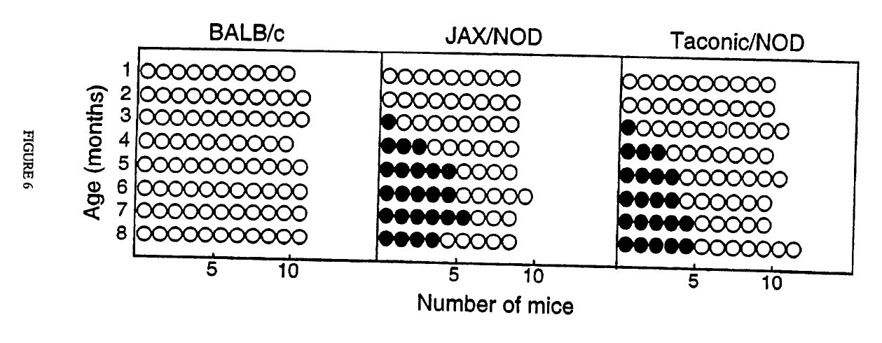

The results are presented in FIG. 6. Once again, RA developed in the F₁ progeny of nondiabetic NOD mice. Twenty-four of 73 (33%) of Jackson Laboratory F₁ females (JAX/NOD) and 22 of the 84 (19%) of Taconic F₁ females (Taconic/NOD) developed ankle swelling indicative of RA.

The results of breeding experiments over a three year time span confirm that the generation of an RA mouse model by breeding a nondiabetic NOD mouse is repeatable and is not limited in scope to a particular parental nondiabetic NOD mouse strain from a particular source or vendor.

### Example 6

### Heritability of RA from P₁ nondiabetic NOD stock

To demonstrate that the RA mouse of the present invention is reproducible in progeny of a P₁ nondiabetic NOD mouse over multiple generations (i.e., F₂, F₃, F₄, etc.), F₁ RA mice (i.e., F₁ NOD-RA mice) generated as described in Examples 1 and 5 were mated to nondiabetic NOD, C57BL/6, and BALB/c mice. In addition, BALB/c mice were mated to C57BL/6 mice as a control group. The incidence of the RA-like disease among multi-generational progeny of NOD-RA mice up to 6 months was determined by ankle thickness.

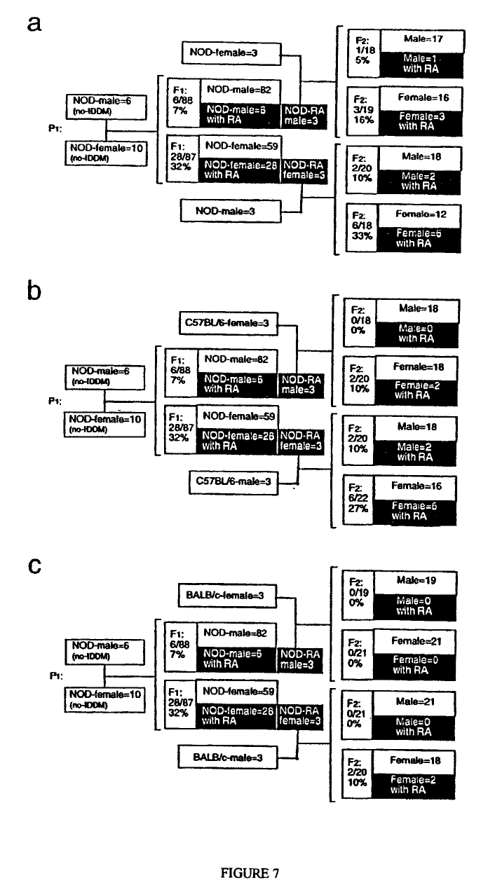

Pedigrees are presented in FIG. 7. All breeding combinations of F1 NOD-RA mice (NOD-RA×NOD, NOD-RA×C57BL/6, and NOD-RA×BALB/c) produced F₂progeny exhibiting symptoms of RA by 6 months of age. RA symptoms were enhanced in female F₂ progeny of NOD-RA females. The rate of disease progression in F₂ NOD/C57BL-RA females after delivery was increased relative to that for virgin F₂ NOD/C57BL-RA females of the same age. None of the F₂ progeny developed diabetes by 30 weeks of age.

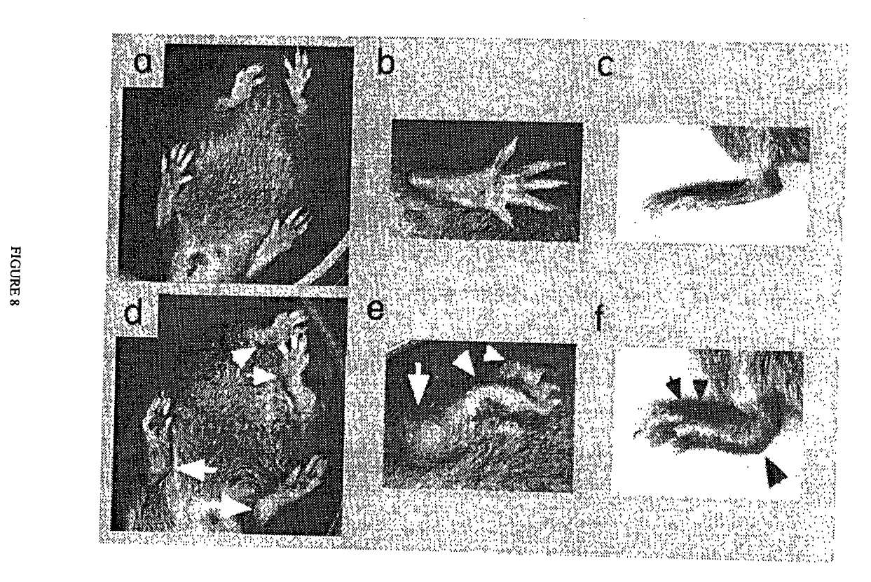

RA was not detected among any progeny from BALB/c×C57BL/6 matings (control data not shown, but see FIGS. 8 a-c), again eliminating a non-specific infection in the colony and confirming a genetic basis as a causal agent for RA among the mice.

FIG. 8 illustrates the gross morphology of the hind feet of a 21-week-old F₂female mouse bred from a BALB/c crossed with a C57BL/6 (FIGS. 8a-c), and the hind feet of a 21-week-old F₂ female mouse bred from a NOD-RA crossed with a C57BL/6 (FIGS. 8d-f). The F₂ NOD-RA progeny showed moderate and symmetrical peripheral fusiform swelling, as well as prominent swelling at the ankle, the metatarsophalangeal and proximal interphalangeal joints, with sparing of the distal interphalangeal joint. Arrows and arrowheads indicate the locations of maximal swelling and joint abnormalities, respectively.

These results confirm that the RA mouse of the present invention may be obtained from progeny of multiple generations descendant from nondiabetic NOD mouse (P₁) stock. The generation of the RA mouse of the present invention is not limited by the mating of a :nondiabetic NOD mouse (or any NOD-RA progeny generated therefrom) to any particular mouse strain. The absence of any RA progeny from the BALB/c×C57BL/6 matings eliminate the possibility of any non-specific causal agent in the colony, and further support earlier findings that a single nondiabetic NOD mouse (of any variety), used as an ancestral parental stock, is all that is required to produce multi-generational NOD-RA progeny.

### Example 7

### Pregnancy Effects of RA in the NOD-RA Mouse

To demonstrate the effects of pregnancy on the onset and progression of RA in the NOD-RA mouse of the present invention, and that these effects mimic those found in human RA, F₁ NOD-RA females were impregnated at 4 months of age and examined weekly during pregnancy and postpartum for indications of RA (as compared to virgin NOD-RA and BALB/c females). In addition, F₂ females produced from these pregnancies were impregnated at 4 months and examined weekly during their pregnancy and postpartum for indications of RA (as compared to virgin F₂ females). Ankle thickness was measured before, during, and after pregnancy in F₁ and F₂ NOD-RA females, as well as in age-matched impregnated BALB/c females and virgin F₁ and F₂ NOD-RA females.

Three to four months following pregnancy, NOD-RA mice showed grossly visible swelling of the ankles, as well as swelling at the metatarsophalangeal and proximal interphalangeal joints of both hind feet. No swelling was observed at the distal interphalangeal joints. In contrast, control BALB/c mice, parental (P₁) NOD mice, and a cohort of 10 NOD females newly obtained from The Jackson Laboratory (with or without diabetes) exhibited normal gross morphology of the peripheral limbs, with no particular soft tissue swelling, when maintained under clean but not pathogen free housing conditions and aging conditions similar to those of the F₁ NOD animals.

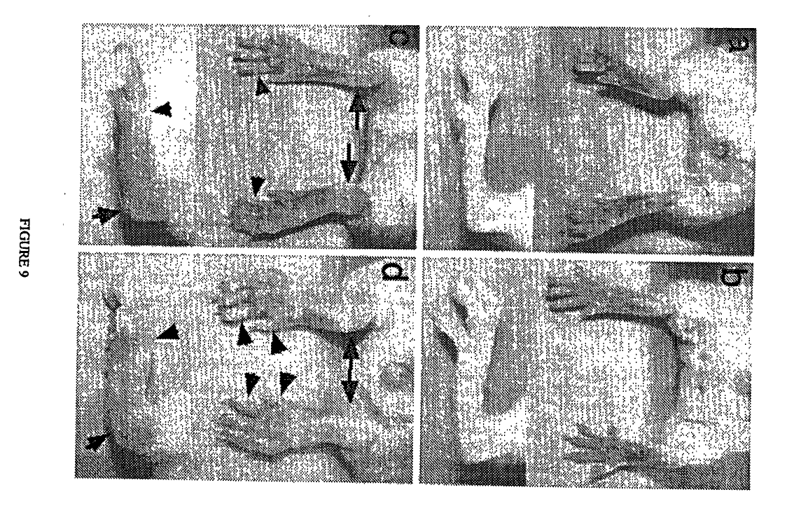

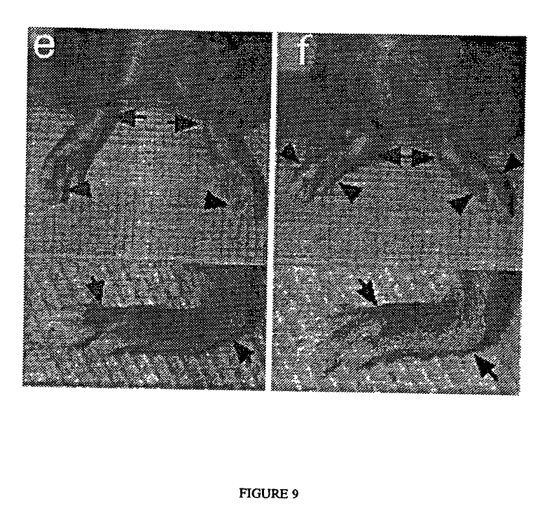

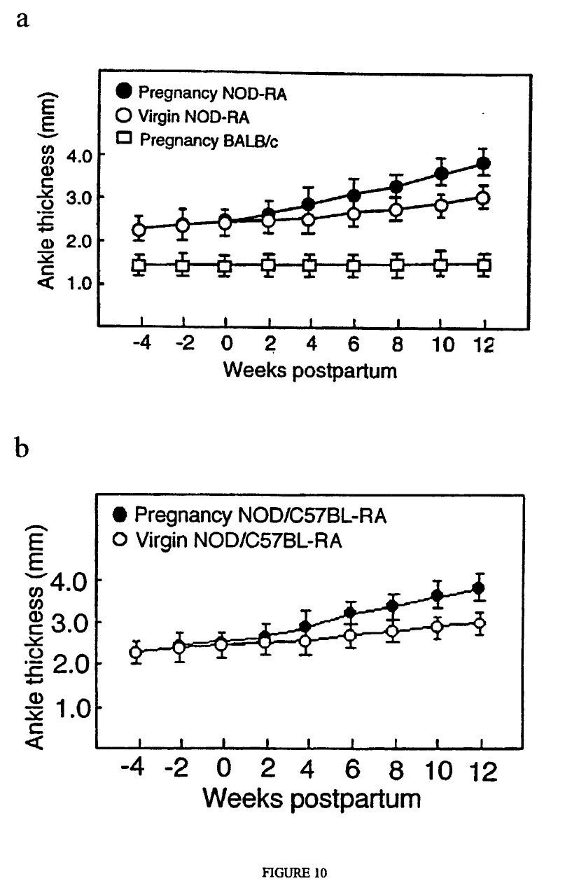

Results from these studies are presented in FIGS. 9 and 10. A pronounced increase in ankle thickness was observed in NOD-RA females compared to the BALB/c and NOD female groups. This swelling remained stable or disappeared altogether during F₁ NOD-RA female pregnancy (arrows indicate maximal swelling and joint abnormalities).

A more rapid progression of RA was observed in the group of NOD-RA females that became pregnant. At eight weeks postpartum, F₁ NOD-RA mothers exhibited marked limb swelling. In contrast, no peripheral swelling was apparent in BALB/c females at the same time after delivery. Furthermore, the rate of disease progression in F₁ NOD-RA mothers after parturition increased relative to virgin F₁ NOD-RA females of the same age (FIG. 10a). The rate of disease progression in F₂ mothers (NOD/C57BL-RA mothers) after delivery also increased relative to virgin F₂ NOD/C57BL-RA females of the same age (FIG. 10b).

These results confirm that pregnancy has a significant impact on RA disease progression and expression in NOD-RA female mice similar to RA manifestations observed in humans. The remission of RA during pregnancy and exacerbation post-partum closely mimics a phenomenon long recognized in human female RA patients.

From the foregoing discussion and data, it can be seen that the NOD-RA mice described herein provide a novel spontaneous model that closely mimics human rheumatoid arthitis. The NOD-RA model exhibits clinical (e.g., radiological, histological, and serological) symptoms that are typical of humans afflicted with rheumatoid ardtritis. Also as with rheumatoid arthritis in humans, the disease in NOD-RA mice is spontaneous and dominant in females, with onset at older ages, incomplete penetrance in a population, lifelong exacerbation after pregnancy. The NOD-RA mouse model may consequently be used advantageously for characterization of the pathogenesis of RA in humans and may be used advantageously in the development and testing of new drugs and diagnostic reagents for treating and detecting or monitoring this prevalent human affliction.

Variations and additional embodiments of the invention described herein will be apparent to those skilled in the art without departing from the scope of the invention as defined in the claims to follow. For instance, although the discoveries related herein pertain to the well-known NOD mouse, other strains of mice that develop autoimmune disease phenotypes, such as SJL mice, NZB mice, and NZW mice, which are prone to develop a lupus erythematosis-like disease, may also show in certain progeny the development of the rheumatoid arthritis-like disease found in the nondiabetic NOD progeny described above. Also, in view of the phenomena described above, common shortcuts such as lymphocyte transfer from NOD-RA mice to naive young NOD mice or, e.g., irradiated mice of other species will be expected to recapitulate the RA seen in the NOD-RA donors. Likewise, other mammalian species commonly used in laboratory experiments, such a guinea pigs and rabbits, which species are susceptible to autoimmune disorders, may be bred and examined in accordance with the discoveries detailed above to establish individuals of another species useful as a model for human rheumatoid arthritis; that is, other mammalian models of autoimmune disease phenotypes may be used as parental stock to produce RA progeny of the present invention for species other than the mouse.

References

Alonzi, T., E. Fattori, D. Lazaro, P. Costa, L. Probert, G. Kollias, F. De Benedetti, V. Poli, and G. Ciliberto. 1998. Interleukin 6 is required for the development of collageninduced arthritis. J Exp Med 187:461.

Andriopoulos, N. A., J. Mestecky, E. J. Miller, and J. C. Bennett. 1976. Antibodies to human native and denatured collagens in synovial fluids of patients with rheumatoid arthritis. Clin Immunol Immunopathol 6:209.

Becker, K. G., R M. Simon, J. E. Bailey-Wilson, B. Freidlin, W. E. Biddison, H. F. McFarland, and J. M. Trent. 1998. Clustering of non-major histocompatibility complex susceptibility candidate loci in human autoimmune diseases. Pro Natl Acad Sci, USA 95:9979.

Becker, K. G. 1999. Comparative genetics of type I diabetes and autoimnuune disease: common loci, common pathways? Diabetes 48:1353.

Bell, C., N. Talal, and P. H. Schur. 1975. Antibodies to DNA in patients with rheumatoid arthritis and juvenile rheumatoid arhritis. Arthritis Rheum 18:535.

Bellamy, N., D. Duffy, N. Martin, and J. Mathews. 1992. Rheumatoid arthritis in twins: a study of aetiopathogenesis based on the Australian Twin Registry. Ann Rheum Dis 51:588.

Bias, W. B., J. D. Reveille, T. H. Beaty, D. A. Meyers, and F. C. Arnett. 1986. Evidence that autoimmunity in man is a Mendelian dominant trait. Amer J Hum Genet 39:584.

Brackertz, D., G. F. Mitchell, and I. R Mackay. 1977. Antigen-induced arthritis in mice. I. Induction of arthritis in various strains of mice. Arthritis Rheum 20:841.

Cavallo, M. G., P. Pozzilli, C. Bird, M. Wadhwa, A. Meager, N. Visalli, A. J. Gearing, D. Andreani, and R. Thorpe. 1991. Cytokines in sera from insulin-dependent diabetic patients at diagnosis. Clin Exp Immunol 86:256.

Clague, R. B., M J. Shaw, and P. J. Holt. 1980. Incidence of serum antibodies to native type I and type II collagens in patients with inflammatory arhritis. Ann Rheum Dis 39:201.

DiCosmo, B. F., D. Picarella, and R. A. Flavell. 1994. Local production of human IL-6 promotes insulitis but retards the onset of insulin-dependent diabetes mellitus in non-obese diabetic mice. Int Immunol 6:1829.

Dinarello, C. A. 1996. Biologic basis for interleukin-1 in disease. Blood 87:2095.

van Eden, W., I. E. Thole, R., van der Zee, A. Noordzij, J. D. van Embden, E. J. Hensen, and I. R Cohen. 1988. Cloning of the mycobacterial epitope recognized by T lymphocytes in adjuvant arthritis. Nature 331:171.

Fantuzzi, G., and C. A. Dinarello. 1998. Interleukin-1β-deficient mice. In Cytokine knockouts. S. C. Durum and K. Muegge, editors. Humana Press, New Jersey. 129.

Feldmann, M., F. M. Brennan, and R. N. Maini. 1996. Role of cytokines in rheumatoid arthritis. Annu Rev immunol 14:397.

Green, E. A., and R. A. Flavell. 1999. Tumor necrosis factor-alpha and the progression of diabetes in non-obese diabetic mice. Immunol Rev 169:11.

Hang, L. M., and R. M. Nakamura. 1997. Current concepts and advances in clinical laboratory testing for autoimmune diseases. Crit Rev Clin Lab Sci 34:275.

Hench, P. S. 1938. The amerliorating effect of pregnancy or chronic atrophic arthritis, fibrositis and intermittent hydroarthrosis. Proceedings of Sl Staff Meetings. Mayo Clinic. 45:161.

Hirano, T., T. Matsuda, M. Turner, N. Miyasaka, G. Buchan, B. Tang, K. Sato, M. Shimizu, R. Maini, and M. Feldmann. 1988. Excessive production of interleukin 6/B cell stimulatory factor-2 in rheumatoid arthritis. Eur J Immunol 18:1797.

Horai, R, S. Saijo, H. Tanioka, S. Nakae, K. Sudo, A. Okahara, T. Ikuse, M. Asano, and Y. Iwakura. 2000. Development of chronic inflanmnatory arthropathy resembling rheumatoid arthritis in interleukin I receptor antagonist-deficient mice. J Exp Med 191:313.

Humphreys-Beher, M. G., Y. Hu, Y. Nakagawa, P. L. Wang, and K. R Purushotham. 1994. Utilization of the non-obese diabetic (NOD) mouse as an animal model for the study of secondary Sjogren's syndrome. Adv. Exp. Med. Biol 350:631.

Iijima, T., H. Tada, A. Yagoro, Y. Hidaka, T. Tomita, T. Ochi, and N. Amino. 1999. Incidence of postpartum onset of disease among patients with rheumatoid arthritis [letter]. J Rheum 26:755.

Jackson, G. 1992. Immunodeficiencies and autoimmune disorder. In Clinical Laboratory Medicine, vol. Chapter 36. R Tilton, editor. Mosby Year Book, Inc., St. Louis, Mo. 485.

Kouskoff, V., A. S. Korganow, V. Duchatelle, C. Degott, C. Benoist, and D. Mathis. 1996. Organ-specific disease provoked by systemic autoimmunity. Cell 87:811.

Lin, J. P., J. M. Cash, Sl. Doyle, S. Peden, K. Kanik, C. I. Amos, S. J. Bale, and R. L. Wilder. 1998a Familial clustering of rheumatoid arthritis with other autoimmune diseases. Hum Genet 103:475.

Lin, L., G. N. DeMartino, and W. C. Greene. 1998b. Coanslational biogenesis of NF-kappa B p50 by the 26S proteasome. Cell 92:819.

Makino, S., K. Kunimoto, Y. Muraoka, Y. Mihima, K. Katagiri, and Y. Tochino. 1980. Breeding of a non-obese, diabetic strain of mice. Jikken Dobutsu. Experi Animals 29:1.

Nicklin, M. J., D. E. Hughes, J. L. Barton, J. M. Ure, and G. W. Duff. 2000. Arterial inflammation in mice lacking the interleukin I receptor antagonist gene. J Exp Med 191:303.

Ohshima, S., Y. Saeki, T. Mima, M. Sasai, K. Nishioka, S. Nomura, M. Kopf, Y. Katada T. Tanaka, M. Suemura, and T. Kishimoto. 1998. Interleukin 6 plays a key role in the development of antigen-induced arthritis. Proc Natl Acad Sci USA 95:8222.

Ostensen, M. 1999. Sex hormones and pregnancy in rheumatoid arthritis and systemic lupus erythematosus. Ann NY Acad Sci 876:131.

Patrick, S. L., C. S. Moy, and R. E. LaPorte. 1989. The world of insulin-dependent diabetes mellitus: what international epidemiologic studies reveal about the etiology and natural history of IDDM. Diabetes-Metab Rev 5:571.

Poli, V. 1998, I1-6 unique functions in inflammation, bone metabolism, and B-cell neoplasias revealed by studies on IL-6-deficient mice. In Cytokine knockouts. S. C. Durum and K. Muegge, editors. 237-258 vols. Humana Press, New Jersey.

Pozilli, P., A. Signore, A. J. Williams, and P. E. Beales. 1993. NOD mouse colonies around the world-recent fts and figures. Immunol Today 14:193.

She, J. X., and M. P. Marron. 1998. Genetic susceptibility ftctors in type 1 diabetes: linkage, disequilibrium and functional analyses. Curr Opin Immunol 10:682.

Silman, A., A. Kay, and P. Brennan. 1992. Timing of pregnancy in relation to the onset of rheumatoid arthritis. Arthritis Rheum 35:152.

Silman, A J., A. l. MacGregor, W. Thomson, S. Holligan, D. Carthy, A. Farhan, and W. E. Ollier. 1993. Twin concordance rates for rheumatoid arthritis: results from a nationwide study. Br J Rheum 32:903.

Taga, T., M. Hibi, Y. Hirata, K. Yanasaki, K. Yasukawa, T. Matsuda, T. Hirano, and T. Kishimoto. 1989. Interleukin-6 triggers the association of its receptor with a possible signal transducer, gp130. Cell 58:573.

Tarkowsli, A., L. Klareskog, H. Carlsten, P. Herberts, and W. J. Koopman. 1989. Secretion of antibodies to types I and II collagen by synovial tissue cells in patients with rheumatoid arthritis. Arthritis Rheum 32:1087.

Turgen, M. L. 1996. Rheumatoid Artritis. 2nd Edition ed. In Immunology and Serology in Laboratory Medicine, vol. Chapter 28. J. Shanahan, editor. Mosby Year Book, Inc., St. Louis, Mo. 387.

Vaughan, J. H. 1993. 1992 Joseph J. Bunim Lecture. Pathogenetic concepts and origins of rheumatoid factor in rheumatoid arthritis. Arthritis Rheum 36:1.

Wolfe, F. 1996. The natural history of rheumatoid arthritis. J Rheum—Supplement 44:13.

Wolffe, A. P., and M. A. Matzke. 2000. Epigenetics: Regulation through repression. Science 286:481.

Wooley, P. H. 1998. Animal models of arthritis, eds. J. H. Klippel and P. A. Dieppe. Mosby Publishers, London. 51 pp.

Wu, J., J. A. Longmate, G. Adamus, P. A. Hargrave, and E. K. Wakeland. 1996. Interval mapping of quantitative trait loci controlling humoral immunity to exogenous antigens: evidence that non-MHC immune response genes may also influence susceptibility to autoimmunity. J Immunol 157:2498.

Yamasaki, K., T. Taga, Y. Hiram, H. Yawata, Y. Kawanishi, B. Seed, T. Taniguchi, T. Hirano, and T. Kishimoto. 1988. Cloning and expression of the human interleukin-6 (BSF-2/IFN beta 2) receptor. Science 241:825.

Zheng, H., D. Fletcher, W. Kozak, M. Jiang, K. J. Hofinann, C. A. Conn, D. Soszynski, C. Grabiec, M. E. Trumbauer, A. Shaw, and et al. 1995. Resistance to fever induction and impaired acute-phase response in interleukin-l betaeficient mice. Immunity 3:9.

All publications cited above are incorporated herein by reference.

1. A mouse exhibiting a symptom of rheumatoid arthritis prepared by the process of:
(a) breeding a male mouse and a female mouse, at least one of which is a nondiabetic NOD mouse; and
(b) selecting a mouse exhibiting a symptom of rheumatoid arthritis from any progeny produced from step (a).

2. A mouse model of rheumatoid arthritis prepared by the process of:
(a) breeding a nondiabetic NOD mouse; and
(b) selecting from any progeny produced from step (a), a mouse that exhibits a symptom of rheumatoid arthritis.

3. A progeny of a nondiabetic NOD female mouse, wherein said progeny exhibits a symptom of rheumatoid arthritis.

4. A progeny of a nondiabetic NOD male mouse, wherein said progeny exhibits a symptom of rheumatoid arthritis.

5. A progeny of a nondiabetic NOD male mouse and a nondiabetic NOD female mouse, wherein said progeny exhibits a clinical symptom of rheumatoid arthritis.

6. The progeny according to any one of claims 1-5, wherein said progeny is a F₁ progeny.

7. The progeny according to any one of claims 1-5, wherein said nondiabetic NOD mouse is a euglycemic NOD mouse at least 6 weeks of age.

8. The progeny according to any one of claims 1-5, wherein said nondiabetic NOD mouse is a euglycemic NOD mouse at least 12 weeks of age.

9. The progeny according to any one of claims 1-5, wherein said nondiabetic NOD mouse is a euglycemic NOD mouse at least 20 weeks of age.

10. A process for producing a mouse, exhibiting a symptom of rheumatoid arthritis, comprising the steps:
(a) breeding a nondiabetic NOD mouse; and
(b) selecting from any progeny produced from step (a), a mouse that exhibits a symptom of rheumatoid arthritis.

11. The process of claim 10, wherein a nondiabetic NOD male mouse is bred with a nondiabetic NOD female mouse.

12. The process of claim 11, wherein the NOD mice of step (a) are approximately the same age.

13. The process of claim 11, wherein the NOD mice of step (a) are approximately the same age and are at least 12 weeks old.

14. The process of claim 11, wherein the NOD mice of step (a) are approximately the same age and are at least 20 weeks old.

15. The process of claim 11, wherein the NOD mice of step (a) are approximately the same age and are 20-24 weeks old.

16. A process for producing a mouse, which exhibits a symptom of rheumatoid arthritis, comprising the steps:
(a) selecting from a population of NOD mice a diabetes-free mouse;
(b) breeding said mouse selected in step (a); and
(c) selecting from any progeny of step (b) a mouse, which exhibits a symptom of rheumatoid arthritis.

17. The process of claim 16, wherein a diabetes-free NOD male mouse is bred with a diabetesfree NOD female mouse.

18. The process of claim 17, wherein the pair of diabetes-free mice are approximately the same age.

19. The process of claim 17, wherein the pair of diabetes-free mice are age-matched and are at least 6 weeks old.

20. The process of claim 17, wherein the pair of diabetes-free mice are age-matched and are at least 12 weeks old.

21. The process of claim 17, wherein the pair of diabetes-free mice are age-matched and are 20-24 weeks old.

22. A process for obtaining RA mice, exhibiting a symptom of rheumatoid arthritis, comprising breeding a diabetes-free NOD mouse and collecting the progeny thereof.

23. A process according to claim 22, wherein at least one of said collected progeny exhibit one or more symptoms selected from the group consisting of: joint and limb swelling, symmetrical enlargement of peripheral joints with sparing of axial skeleton (e.g., hips, spine), spontaneous rupture of extensor tendons, Boutonniere deformity, synovitis, leukocyte invasion of the pancreas, pannus formation, cartilage destruction, bone degeneration, autoantibodies specific for the Fc region of immunoglobulin G, and increases in IL-6 in serum.

24. The process according to claim 22, wherein said diabetes-free NOD mouse is characterized as diabetes-free by a method selected from the group consisting of:
(a) survival to 20-28 weeks of age;
(b) survival to at least 20 weeks of age without development of hyperglycemia;
(c) levels of serum IL-6 not significantly elevated in comparison to normal mice;
(d) absence of leukocyte infiltration of the pancreas;
(e) absence of rheumatoid factor in serum; and
(f) production of offspring having symptoms of rheumatoid arthritis.

25. A method for isolating an agent useful for treatment of rheumatoid arthritis comprising:
administering an agent to a mouse according to any of claims 1-5 and determining whether there is reversal or improvement in any of the rheumatoid arthritis symptoms exhibited by said mouse.

26. A method for isolating an agent useful for prevention of rheumatoid arthritis comprising:
administering an agent to one or more progeny of a nondiabetic NOD mouse and observing, with respect to said progeny, penetrance of rheumatoid arthritis symptomology, onset of any rheumatoid arthritis symptomology, or the magnitude of rheumatoid arthritis symptoms exhibited in any of said progeny, wherein an agent associated with decreased penetrance, delayed onset, or reduced symptoms indicates usefulness of the agent for prevention of rheumatoid arthritis.
